(19) United States
(12) Patent Application Publication
(10) Pub. No.: US 2003/0135268 A1
(43) Pub. Date: Jul. 17, 2003

(54) Secure stent for maintaining a lumenal opening

(76) Inventor: Ashvin Desai, San Jose, CA (US)

Correspondence Address: David H. Jaffer, Pillsbury Winthrop LLP, 2550 Hanover Street, Palo Alto, CA, 94304-1115, US

(21) Appl. No.: 09/547,708

(22) Filed: Apr. 11, 2000

(51) Int. Cl. A61F 2/06

(52) U.S. Cl. 623/1.19; 623/1.15

(57) ABSTRACT

A secure stent for maintaining a lumenal opening constructed preferably as a tubular structure of NiTi material or bioabsorbable polymers. The circumference of the tube is preferably in the shape of a polygon in contrast to the circular or oval shape of a body lumen into which the stent is to be placed. The polygon shape and ribs provides interference with the lumen wall and resists stent migration. The diameter of the stent tube is configured with each end enlarged providing flanges for interference with a lumen wall. The central portion of the stent is also bulged out to an increased diameter to provide an enhanced lumen wall resistance to avoid migration. In addition, the locking feature of a ribbed structure prevents the stent from collapsing, and thereby maintains the lumen opening. The stent is preferably constructed from polymers, including bioabsorbable polymers, and/or super elastic materials. The bioabsorbable polymer construction aids removal by causing the tube diameter to collapse. Removal of the stent can therefore be accomplished by simply grasping the proximal end of the stent. Alternatively, a stent constructed entirely of bioabsorbable material will eventually be entirely absorbed, avoiding the need for removal. Alternatively, the stent can be preferably constructed of NiTi or other shape memory material and set in the desired shape at a high temperature. Installation is accomplished by cooling the stent to the malleable Martensite state and winding it on a small diameter mandrel of an insertion/removal tool. The compacted stent is then placed in a probe and inserted in a body lumen, whereupon it is heated to an Austenite state where it regains its spring tension, forcing it back toward the set shape. Removal is accomplished by cooling the stent to the malleable Martensite state and pulling it out. If the selected material is bioabsorbable, the stent generally does not have to be removed.

### BACKGROUND OF THE INVENTION

1. Field of the Invention

The present invention relates generally to intralumenal stent devices for maintaining a lumenal opening in a human body, and more particularly to a urethral stent that is configured to keep open the lower urinary tract and other body lumens.

2. Description of the Prior Art

Various devices known as stents have been proposed, developed and used for placement in a human body to maintain a lumen opening. Typical applications include treating occlusions of blood vessels, and urethra blockages due to benign prostate hyperplasia. Problems that generally need attention in the design and use of stents include methods of insertion and removal, and prevention of stent migration. Most stents in the marketplace are constructed of a metallic coil of nitinol alloy or stainless steel. In U.S. Pat. No. 5,830,179 a stent is constructed as a coil of nitinol alloy. Nitinol is a member of a class of materials known to have “shape memory.” In practice, the wire is heated to a high temperature, wound on a mandrel or otherwise placed in a set position and cooled. The material stresses result in a “spring” tension built into the material to return to the set position as long as the material is above a certain temperature known as an Austenite state. In order to insert the stent in a body lumen, it is cooled, causing it to enter what is known as a Martensite state in which it is very malleable and can be wound on a small diameter mandrel. Once in position in the body lumen, the stent is heated, resulting in its entering back into the Austenite state, wherein the spring tension is restored, urging it back toward the set position. An alternate design uses outwardly flanged ends to provide increased resistance with the lumen wall.

### SUMMARY

It is therefore an object of the present invention to provide an improved stent that can be readily removed.

It is a further object of the present invention to provide a stent that effectively resists migration after installation.

It is another object of the present invention to provide a stent that has a coating for delivery of a treatment substance.

Briefly, a preferred embodiment of the present invention includes a secure stent for maintaining a lumenal opening constructed preferably as a tubular structure of NiTi material or bioabsorbable polymer. The circumference of the tube is preferably in the shape of a polygon in contrast to the circular or oval shape of a body lumen into which the stent is to be placed. The polygon shape and ribs provide interference with the lumen wall and resist stent migration. The diameter of the stent tube is configured with each end enlarged providing flanges for interference with a lumen wall. The central portion of the stent is bulged out to an increased diameter to provide an enhanced lumen wall resistance to avoid migration. In addition, the locking feature of a ribbed structure prevents the stent from collapsing, and thereby maintains the lumen opening. The stent is preferably constructed from polymers, including bioabsorbable polymers, and/or super elastic materials. The bioabsorbable polymer construction aids removal by causing a reduction in the tube diameter as material is absorbed by body material. Attachment for removal of the stent can then be accomplished by simply grasping the proximal end of the stent. Alternatively, a stent constructed entirely of bioabsorbable material will eventually be entirely absorbed, avoiding the need for removal. Alternatively, the stent can be constructed of NiTi or other shape memory material and set in the desired shape at a high temperature. Installation is accomplished by cooling the stent to the malleable Martensite state and winding it on a small diameter mandrel of an insertion/removal tool. The compacted stent is then placed in a probe and inserted in a body lumen, whereupon it is heated to an Austenite state where it regains its spring tension, forcing it back toward the set shape. Removal is accomplished by cooling the stent to the malleable Martensite state and pulling it out. If the selected material is bioabsorbable, the stent generally does not have to be removed.

### IN THE DRAWING

FIG. 1a contains side and end views of a preferred embodiment of the stent of the present invention;

FIG. 1b shows an alternate embodiment with a circular end view;

FIG. 2a shows a stent with a hexagonal cross section of constant area;

FIG. 2b shows a hexagonal stent with a concave central section;

FIG. 2c shows a stent with a hexagonal cross section and convex/bulbous central section;

FIG. 3 shows a stent formed from perforated, thin flat material;

FIG. 4a is a view of flat, stepped material for forming a stent;

FIG. 4b shows the stepped material formed in an expanded spiral;

FIG. 4c shows the stepped material in a tight, compact form;

FIG. 5a is a perspective view of an expanded stent constructed with narrow, flat protrusions;

FIG. 5b shows the stent of FIG. 5a wound in a compact form;

FIG. 6a shows a stent similar to FIG. 5a with a corrugated elongated protrusion;

FIG. 6b shows the stent of FIG. 6a wound in a compact form;

FIG. 7a shows sharply and evenly corrugated sheet material;

FIG. 7b shows a stent wound from the corrugated material of FIG. 7a;

FIG. 7c shows a stent having an alternate wound form, and constructed from the material of FIG. 7a;

FIG. 7d shows a stent wound from material with alternating abrupt and tapered lengths;

FIG. 7e illustrates a stent wound from a corrugated material with abrupt points separated by curved sections;

FIG. 7f shows a stent wound from continuously curved corrugated material;

FIG. 7g illustrates holes in stent material;

FIG. 8 illustrates the use of a turn block to expand and contract the cross section of a stent;

FIG. 9 shows a scissor-jack for expanding and contracting a stent;

FIG. 10 illustrates the use of a biodegradable coating over a stent base;

FIG. 11 is a list of bio-absorbable/biodegradable materials;

FIG. 12 is a list of anti-microbial coating materials;

FIG. 13 lists coating materials that can be used as lubricants;

FIG. 14 is a list of drugs/pharmaceuticals, etc. for inclusion in a stent coating;

FIG. 15 shows a stent base of smaller diameter with perforations through which a material can be ejected to secure the stent base to a body lumen wall;

FIG. 16 illustrates a stent in the form of a balloon;

FIG. 17a illustrates an endoscopic instrument for inserting a stent;

FIG. 17b is an expanded view of a stent and ejection device in reference to FIG. 17a;

FIG. 18 shows a polycatheter and balloon device for inserting a stent; and

FIG. 19 illustrates a simple stent installation tool.

### DETAILED DESCRIPTION OF THE PREFERRED EMBODIMENT

A preferred embodiment of the present invention is illustrated in FIG. 1a wherein a tubular stent 10 formed from coiled wire 11 is shown in a longitudinal view 12 and an end view 14. The present invention includes longitudinal variations in the stent cross section, the longitudinal direction defined by axis 16. The stent 10 has a flared proximal end 18 and a flared distal end 20. The middle portion 12 is bulged out. The combination of these variations in the cross section, i.e. variations in the distance of the tube wall from axis 16 as a function of distance along the axis 16, including flared ends 18, 20 and the bulged midportion 22 results in a stent with an increased strength to retain a lumen wall, and an increase in resistance to stent migration/movement in a body lumen. The bulged central/midportion 22 is important in that it provides greater strength in resisting lumen wall pressure than a straight tube section would provide. The stent wall can be constructed from any of various biologically compatible materials, such as NiTi, stainless steel, and various biodegradable polymers. The benefit of the construction is that pressure on the rim 24 of the flares 18, 20 is transferred in part to bulbous midsection 22, giving it greater strength. The end view 14 illustrates another feature of the present invention, showing the outline of the rim 24 of the distal end 20 of the flare. This hexagonal shape continues for the entire length of the stent 10, varying in area from a maximum at the ends 18, 20 and in the middle 22 to a minimum contour 26 between the midsection and flared ends. The hexagonal shape is a preferred embodiment, but other irregular shapes are also included in the spirit of the present invention. The novel purpose of an irregular outline for a stent is to provide increased frictional contact with a typically round or oval shaped lumen wall. The pressure of the irregular shaped stent against the lumen wall causes the wall to expand and partially conform to the stent outline. The irregular shaped stent contour provides areas (for example at 28) of increased pressure, resulting in more resistance with the body lumen wall than would occur if the stent outline were round or oval, such as illustrated in the alternate embodiment of FIG. 1b.

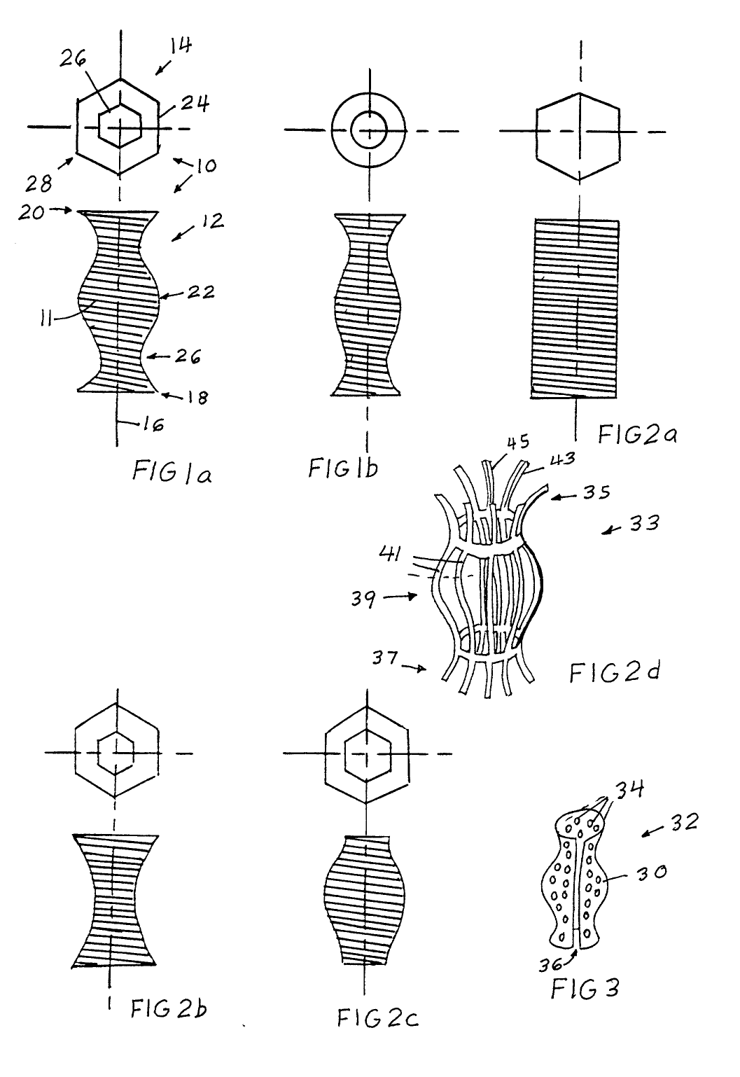

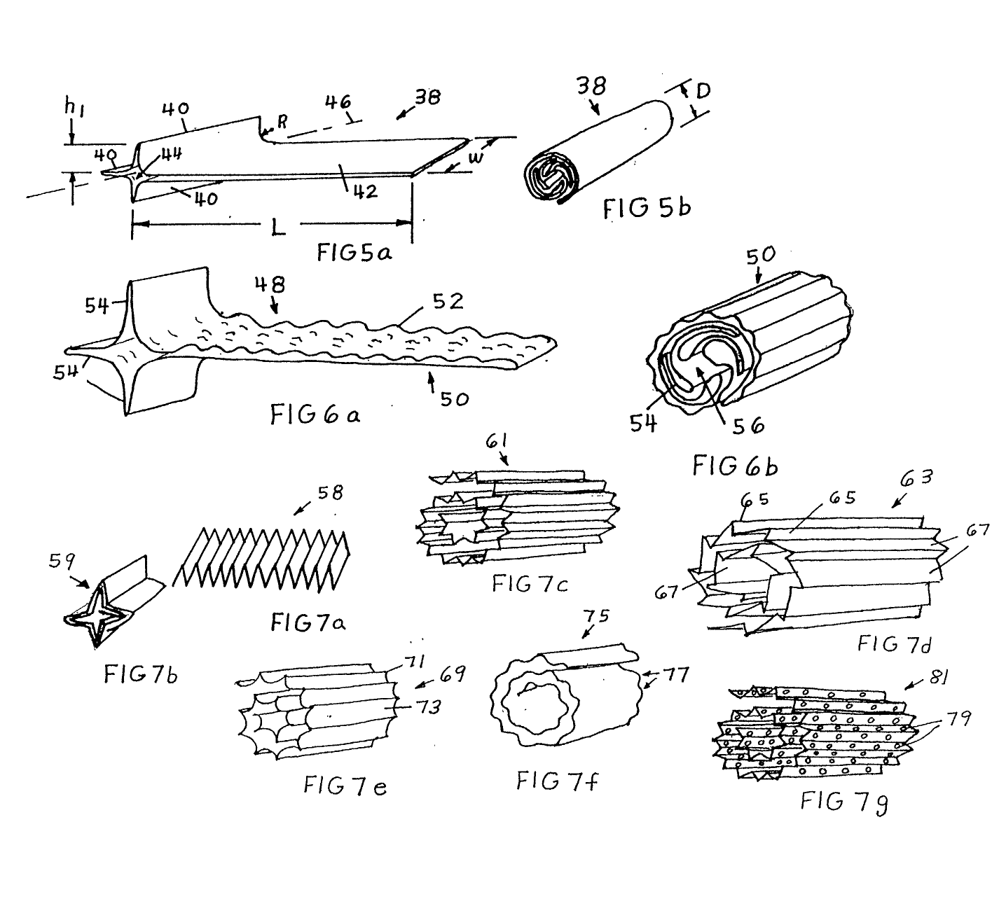

FIG. 2a shows an embodiment utilizing only the irregular cross-section feature, without the variation in cross-section over the length of the stent. FIG. 2b illustrates the use of the irregular cross-section combined with flared ends. FIG. 2c shows a stent with only a bulged middle. FIG. 2d shows a stent 33 formed by slotting the stent material on each end 35, 37 and in the middle 39, and then expanding the ribs 41 outward. The ribs 41 can be a flat ribboned shape or as generally shown, or they can be further configured as illustrated with rib end sections 43 and 45 that are bent outward longitudinally to provide a sharper rib shape, such as the ribs shown in FIGS. 6b, 7c, etc.

FIG. 3 shows the use of a thin sheet material 30 to form a stent 32. The perforations 34 are optional. The slot 34 allows the stent 32 to be readily collapsed for insertion and removal.

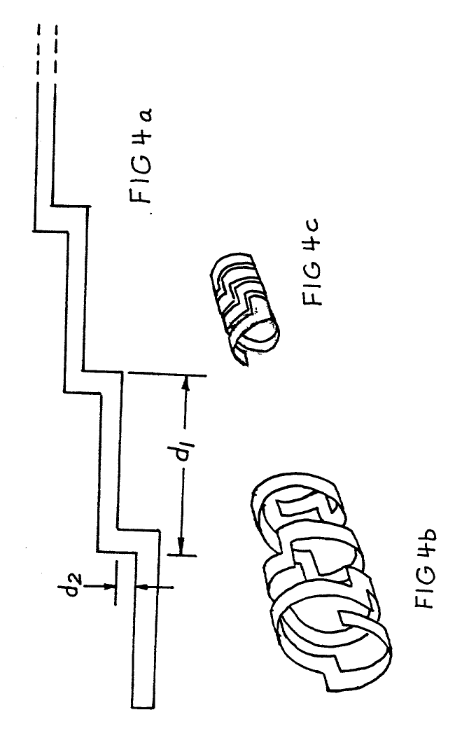

FIGS. 4a-4c illustrate a stent construction using a flat ribbon type of material that is cut in steps as shown in FIG. 4a. The steps are therefore formed in the plane of the flat, sheet/ribbon material as distinguished from steps or corrugations that will be shown in subsequent figures of the drawing. When the material of FIG. 4a is wound on a mandrel, it has an expanded form as shown in FIG. 4b. It can be heated and set in the expanded configuration of FIG. 4b, and then compressed by further winding to a smaller configuration such as FIG. 4c. The step lengths “d₁” determine the minimum circumference of the tightly wound stent as shown in FIG. 4c. Each “turn” of the stent is spaced from the next by the distance d₂ which can be any value desired.

FIGS. 5a and 5b illustrate another alternate stent 38 embodiment. Constructed from flat material of width “w”, it is bent, forming a plurality of short protrusions 40 of lengths hi and a single elongated protrusion 42 of length L. The protrusions 40 and 42 are joined with a radius R, allowing an open lumen 44 through the full length of the stent which is the width “w” of the flat material. The stent 38 is placed in a cylindrical shape as shown in FIG. 5b by winding the elongated protrusion 42 around the axis 46 of the stent lumen 44, in the process folding/bending over the short protrusions 40 resulting in a compressed stent 38 of small diameter D for insertion into a body lumen. FIGS. 6a and 6b illustrate an alternate embodiment 48 of the same general type as shown in FIGS. 5a and 5b. he elongated protrusion 50 has a corrugated side 52 that is included to increase contact resistance with a body lumen wall to reduce stent migration. FIG. 5b shows the compressed, wound state of the stent, clearly showing the corrugated side 52 facing outward. This figure also clearly illustrates bent shorter protrusions and a stent lumen 56 that are features in common with the stent 38 of FIGS. 5a and 5b.

A further alternate stent embodiment 58 is shown in its wound compressed state in FIG. 7a. It is formed from a corrugated material 59 as shown in FIG. 7b. Additional alternate stent embodiments constructed from corrugated sheet material are shown in FIGS. 7a-7g. Shown in FIG. 7a is an evenly bent material 58 which can be wound to form stents 59 and 61 as shown in FIGS. 7b and 7c.

FIG. 7d shows a similar stent 63, differing from stent 61 in that the sheet material is bent so as to provide abrupt ridges 65, which interfere with each other to resist winding once the stent 63 is expanded, providing a self-locking feature.

In fact, all of the stents of FIGS. 7b-7f provide a degree of resistance to compression/rewinding due to the resistance provided by interfering corrugations. Stent 75 of FIG. 7f provides the least resistance, having smoothly formed corrugations. Expansion is encouraged in the stent 61 design of FIG. 7d by the more gently sloping ramps 67.

The stent 69 of FIG. 7e uses ridges 71 separated by curved portions 73. In FIG. 7f the stent 75 is constructed of continuously curved corrugations 77. Any of the stents constructed of sheet material can also have holes, such as holes 79 in stent 81 of FIG. 7g.

The stents of FIGS. 1-7 are preferably constructed of a shape memory material and heat set in an expanded configuration in the Austenite state. In order to insert the stent in a body lumen, it is cooled to the Martensite state wherein the material becomes malleable, lacking resiliency. In this state, the material can be reformed to a compact state. In this compact state, it can be inserted into a body lumen. The stent is preferably placed on a mandrel that is part of an insertion tool prior to cooling and compacting.

A preferred shape memory material is nitinol (NiTi), but the present invention includes the use of other shape memory materials that will be apparent to those skilled in the art. In addition, the stent material can be a biodegradable material, such as a biodegradable polymer. The stents can also be made from a combination of biodegradable and non-degradable materials. For example, in FIG. 7f, the outer layer can be constructed from a biodegradable material, and the inner layer can be constructed of a non-biodegradable material. In this case, when the outer layer is absorbed, the inner layer can be removed.

The stents can also be constructed from nitinol or other super elastic material, processed/heat-treated to what is known as a “super elastic” state. In this state the material retains its resiliency at lower temperatures, and can be used for a permanent stent installation. Removal would require use of a tool to cut or compress the stent.

The stents of FIGS. 1a through 2c, and FIGS. 4b, 5a, 6a and 7b are all shown in an expanded state. When they are constructed of a shape memory material and heat set in this expanded state, they can then be cooled and wound or otherwise compressed to minimize the size during insertion into body lumen. The shape of FIGS. 4c, 5b, and 6b are all examples of a compressed stent in its Martensite state. After insertion in a body lumen, the temperature rises and the material returns to the Austenite state, regaining its resiliency, and causing a force against a body lumen wall in the effort to return to the original state. The stent 58 as shown in FIG. 7a can conceivably be further compressed for insertion by bending the protrusions.

As mentioned above, the stents as disclosed herein can be made out of any bio-compatible material that will allow some method of insertion and removal from a body lumen. The stents of FIGS. 1-7 could be constructed of a permanently resilient material such as stainless steel, and could be collapsed with some difficulty for installation in a probe for insertion. However, removal in such a case would generally require a forceps. Constructing the stents of FIGS. 1-7 with a shape memory material as discussed above is preferred. After cooling the stent, it can be wound, folded, collapsed, etc. as required in order to be loaded into a probe lumen for transport into a body lumen. A push rod in back of the stent in the probe lumen can be used to eject the stent once the probe is in the desired location. As discussed above, the stent is then simply heated, by any of various means including body temperature or a warm saline solution to bring the stent back to the Austenite state wherein it regains its original resiliency. Removal is accomplished by injecting a cool saline solution to bring the stent back to the malleable Martensite state, whereupon it can be readily pulled out.

The flared ends 18, 10 of FIG. 1 provide resistance with the body lumen wall, keeping the stent from moving in the lumen. The bulbous portion 22 is placed where maximum body lumen enlargement is required. The force of the body lumen on the stent portion 22 tends to cause the ends 18 and 20 to expand, which provides enhanced resistance with the lumen walls to avoid migration. The force of the ends 18 and 20 on the body lumen wall is also reflected back to provide resistance to compression of portion 22.

Other shapes for the circumference/cross-section of the stents are also included in the spirit of the present invention. For example, the polygon shape in FIG. 1 could be square, five-sided, an octagon as shown, etc., or other irregular shape to increase resistance between the stent and the body lumen wall. The present invention also includes a circular or oval circumference, as indicated in FIG. 1b.

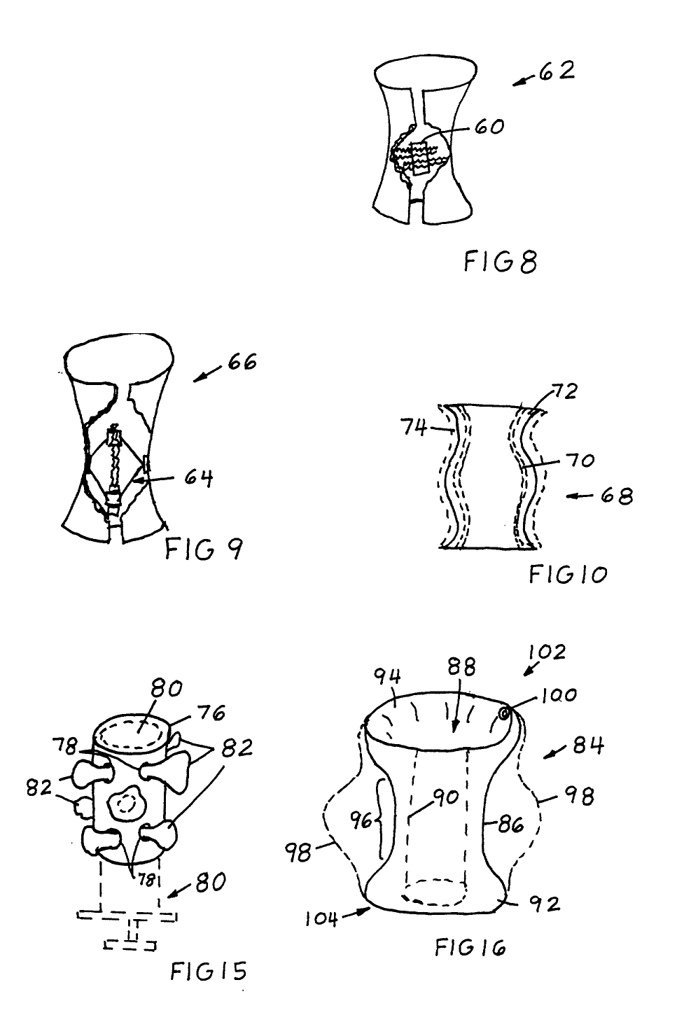

An alternate method of collapsing and expanding a stent is illustrated in FIGS. 8 and 9. An expansion and contraction apparatus can be installed inside a stent that is constructed of sheet material. FIG. 8 shows a turn block 60 that can be activated to contract the diameter of a stent 62 for insertion in a body lumen. The turn block can then be applied to expand the stent against the lumen wall. When removal is required, the reverse procedure is applied. FIG. 9 shows a similar arrangement where a scissor apparatus 64 (similar to a small car jack) is used to expand and contract the diameter of a stent 66.

FIG. 10 illustrates another embodiment of a stent 68 that is designed for temporary use. NiTi or other biologically compatible material 70 is used to form a stent base. A coating of biodegradable material 72 is placed over the base 70. The base can optionally also be made of biodegradable material. The base 70 can be of any desirable configuration that can be collapsed for insertion in a probe lumen for installation in a body lumen, including structures similar to those of FIGS. 1-7. The expanded size of the base 70 is preferably small enough to clear the size of the body lumen into which it is to be placed, if it is not biodegradable, so that when the material 72 is absorbed by the body, the base 70 can be easily removed. A second coating 74, or first coating if coating 72 is omitted, can be included. The coating 74 is generally for inclusion of some type of treatment substance, but can be for any purpose, including the purpose of providing interference with the body lumen walls. If coatings 72 and/or 74 are for determining the stent size, the selection of material 72 and thickness depend on how long the stent is to remain in the body. After the material has been sufficiently absorbed, the stent base can be removed by simply grasping it with an appropriate device through an endoscope lumen, for example. This procedure avoids the need to compress the stent for removal, although collapsible stents can also be used. If the stent base is biodegradable, it will eventually be absorbed, and may therefore not have to be removed.

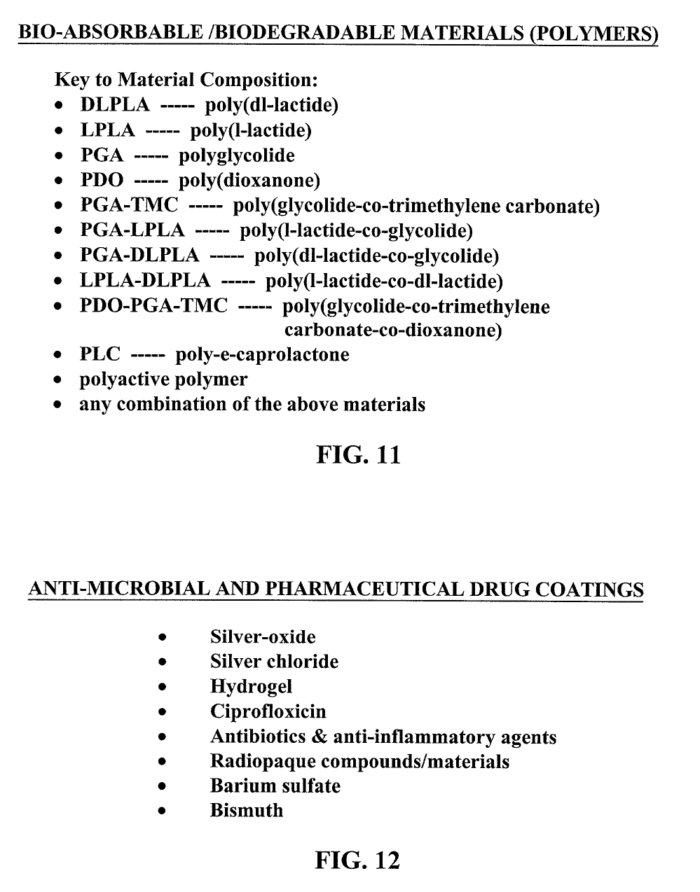

FIG. 11 lists various biodegradable materials that can be used to coat a stent, as indicated above. The stents, or stent bases, described above can be constructed from NiTi or a biodegradable polymer, or any other appropriate material known to those skilled in the art, such as stainless steel or any of various compatible polymers. As mentioned above, stents can be constructed entirely from biodegradable material. A number of these are listed in FIG. 11 and will be recognized by those skilled in the art as applicable to the construction of the stent designs disclosed herein. The benefit of using an all biodegradable material is to avoid the necessity of any removal of a stent.

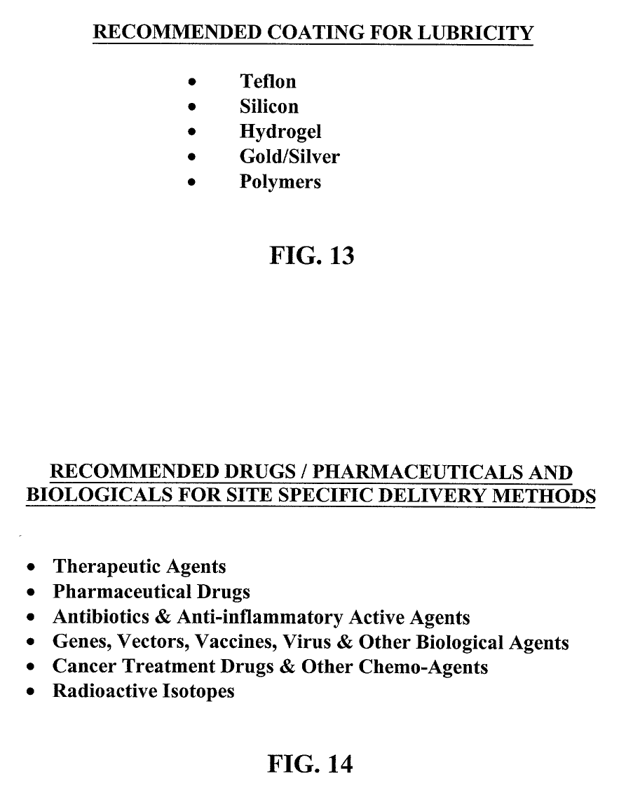

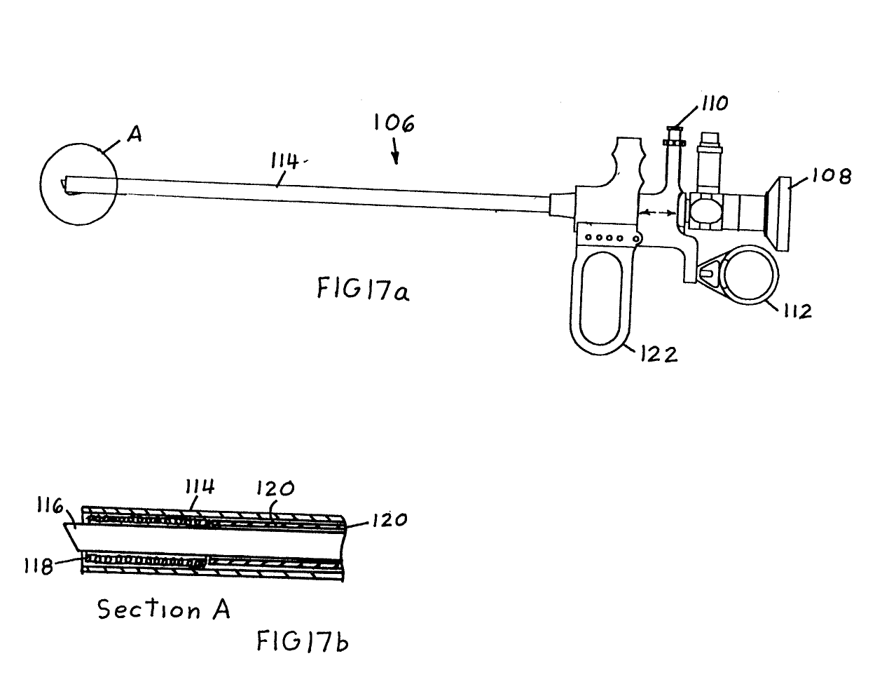

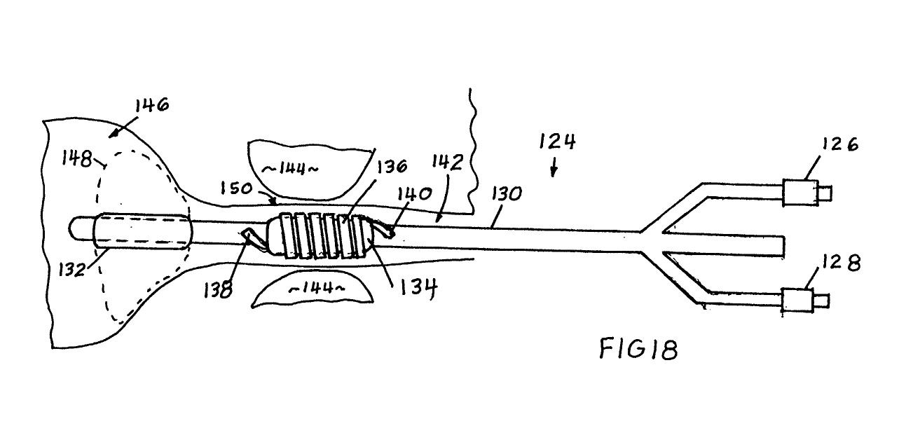

Coating materials for layer 74, as discussed above in reference to FIG. 10, include, but are not limited to those listed in FIGS. 12, 13 and 14. FIG. 12 lists anti-microbial coating materials to reduce the possibility of infection. FIG. 13 lists a selection of materials that reduce friction, i.e. for lubrication. FIG. 14 lists various drugs/pharmaceuticals, etc. as examples of potentially beneficial materials that can be applied to the stent to provide a localized treatment of body tissues.

Another embodiment of the present invention is illustrated in FIG. 15 wherein a cylindrical stent base 76 includes a plurality of holes 78. The stent base 76 is inserted in place in a body lumen. An injector probe, symbolically illustrated as item 80, is then inserted inside the base 76, and a bio-compatible material 82 is injected and forced out the holes 78 and against the wall of the body lumen (not shown). The material 82 would preferably be constructed to harden i.e. set-up quickly after injection.

A still further embodiment of the present invention is illustrated in reference to FIG. 16 wherein an inflatable balloon is used as a temporary stent. The balloon 84 is shown in its uninflated state by solid lines 86. The balloon has a lumen 88 therethrough. The walls of the balloon are constructed with variations of thickness to force expansion in desired directions. The wall 90 of the lumen 88, and the walls of the flared end sections 92, 94 are thicker to avoid expansion. The wall 96 of the outside center portion is thinner to force expansion upon balloon inflation, the inflated state indicated by the dashed lines 98. A self-sealing inflation port 100 is provided on a proximal end 102. The distal end 104 is inserted first in the body lumen.

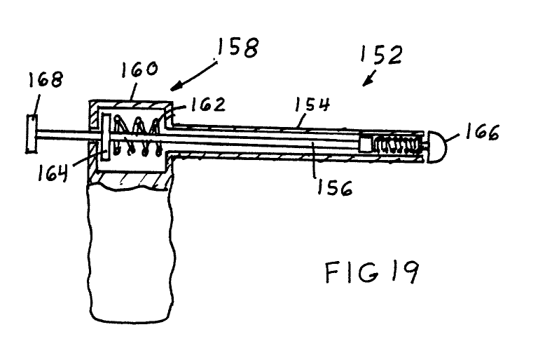

A method and apparatus for inserting a stent is illustrated in FIGS. 17a and 17b utilizing an endoscopic instrument 106. The instrument 106 has a telescope 108, an irrigation/aspiration port 110, and a slide trigger 112. Section “A” of FIG. 17a is shown enlargened in FIG. 17b. The endoscopic instrument 106 includes a probe 114, in which is inserted a first tube 116 through which a telescope probe and/or instrument can be passed. A stent 118 is assembled over the first tube 116. In order to eject the stent from the tube 116, a second tube 120 is provided encircling the first tube 116. The second tube 120 is linked to the slide trigger 112 and pressing the trigger 112 in toward handle 122 moves the second tube 120 to eject the stent from the probe 114. With the stent in place in the body lumen, it will immediately expand if it is constructed from a permanently resilient material such as stainless steel. If it is constructed from a shape memory material such as Nitinal (NᵢTᵢ), it would have been cooled prior to insertion and compressed in the Martensite state. When it is in place in the body lumen, it expands when its temperature is raised, bringing it back into the Austenite state and regaining its resiliency. Removal of the shape memory material is accomplished by cooling the stent to bring it into the maleable Martensite state and then simply pulling it out.

FIG. 18 illustrates another tool that can be used to insert a stsent, including a polycatheter 124 having two one way valves 126 and 128. The catheter 124 has a probe 130 upon which is mounted a first balloon 132 supplied with air by way of valve 126 and a second balloon 134 supplied with air by way of valve 128. A stent 136 is positioned around the second balloon 134. The stent 134 shown in FIG. 18 is formed from flat ribbon material for ease of illustration, but any of the stents disclosed above in FIGS. 1-4, as well as others that will be apparent to those skilled in the art can be installed. The ends 138, 140 of the stent material are releasably attached to the probe 130. The method of attachment can be through use of an adhesive, or by way of other fragile connection.

Insertion of the stent 136 is illustrated in FIG. 18, with the catheter inserted in a urinary tract 142 and the stent 136 positioned adjacent the prostate 144. The first balloon 132 is positioned just inside the bladder 146 at this point in the procedure. Air is applied to the first balloon 132 through one way valve 126, expanding it as shown by dashed lines 148 to contact the bladder wall, securing the catheter 124 in place. The second balloon is then inflated through valve 128. As the balloon 134 expands, tension is placed on the attachment of the ends 138 and 140 until they break, freeing the stent 136 to expand against the wall 150 of the urinary tract 142. If the stent is stainless steel or other permanently resilient material, it will immediately expand upon breaking the attachment ends 138 and 140. If the stent is a shape memory material, it will expand after first being raised in temperature to the Austenite state, which may occur from body temperature or by injection of a heated solution into the urinary tract.

FIG. 19 is a simplified sketch for illustrating some of the features of a stent insertion tool. The tool 152 includes a body probe 154 for insertion in a body lumen, and an installation probe 156. An apparatus 158 includes a housing 160, a spring 162, and plate 164 attached to the probe 156, all configured to apply a spring force to retain the probe 156 inside probe 154 during traversal of a body lumen. With the probe head 166 in place, an operator pushes on the button 168, impelling the stent as explained above. Upon releasing the button, the spring 162 retracts the probe 156.

Although the present invention has been described above in terms of specific embodiments, it is anticipated that alterations and modifications thereof will no doubt become apparent to those skilled in the art. It is therefore intended that the following claims be interpreted as covering all such alterations and modifications as fall within the true spirit and scope of the invention.

It is claimed that:

1. An apparatus for maintaining a body lumen opening comprising a stent in the form of a tube having an axis and having a flared distal end and a flared proximal end and a bulbous middle section.

2. An apparatus as recited in claim 1 wherein a cross-sectional view of said tube orthogonal to said axis shows an irregular shape of said wall.

3. An apparatus as recited in claim 2 wherein said shape is a polygon.

4. An apparatus as recited in claim 3 wherein said polygon is a hexagon.

5. An apparatus for maintaining a body lumen opening comprising a stent in the form of a tube having a structure defining a tube wall and having an axis, wherein a cross-sectional view of said tube wall orthogonal to said axis shows an irregular shape of said wall.

6. An apparatus as recited in claim 5 wherein said shape is a polygon.

7. An apparatus for maintaining a body lumen opening comprising a stent in the form of a tube including a flexible wall, and having a wall adjustment apparatus for expanding and contracting a diameter of said tube.

8. An apparatus as recited in claim 7 wherein said adjustment apparatus includes a turn block.

9. An apparatus as recited in claim 7 wherein said adjustment apparatus includes a scissor jack.

10. An apparatus as recited in claim 1 wherein said tube is in the form of a balloon.

11. An apparatus as recited in claim 10 wherein said structure includes an inner wall defining a lumen through said tube, and said structure having an outer wall that expands upon inflation of said balloon to form said bulbous middle section.

12. An apparatus for maintaining a body lumen opening comprising a stent in the form of a tube having a tube wall, and said wall having a plurality of openings for ejection of material forced from an applicator probe inserted on an inside of said tube, said material ejected from said openings for providing interference with a body lumen wall in which said tube is placed and for prevention of migration of said stent.

13. An apparatus as recited in claim 1 wherein said stent further includes a coating of material on an outside of said tube wall.

14. An apparatus as recited in claim 13 wherein said coating is biodegradable.

15. An apparatus as recited in claim 14 wherein said coating is for the purpose of retaining said stent in a body lumen, and wherein said stent tube can be removed upon degradation of said material.

16. An apparatus as recited in claim 5 wherein said stent further includes a coating of material on an outside of said tube wall.

17. An apparatus as recited in claim 16 wherein said coating is biodegradable.

18. An apparatus as recited in claim 17 wherein said coating is for the purpose of retaining said stent in a body lumen, and wherein said tube can be removed upon degradation of said material.

19. An apparatus as recited in claim 7 wherein said stent further includes a coating of material on an outside of said tube wall.

20. An apparatus as recited in claim 5 wherein said stent is formed of sheet material.

21. An apparatus as recited in claim 20 wherein said stent is constructed by winding a length of said sheet material, wherein said length is a stepped configuration in a plane of said sheet material.

22. An apparatus as recited in claim 20 wherein said sheet material is in the form of a ribbon.

23. An apparatus as recited in claim 22 wherein said material is in the form of said ribbon bent in corrugations.

24. An apparatus as recited in claim 22 wherein said ribbon is bent to from a plurality of short protrusions and a single long protrusion, that is longer than said short protrusion, and said long protrusion is bent around said short protrusions.

25. An apparatus as recited in claim 23 wherein said ribbon is wound to form a plurality of turns.

26. An apparatus as recited in claim 25 wherein, upon compression of said stent, said corrugations of one turn interfere with corrugations of an adjacent turn to resist collapse of said stent, thereby said stent incorporating a self-locking feature.

27. An apparatus as recited in claim 5 wherein said stent structure includes a super elastic material.

28. An apparatus as recited in claim 1 wherein said stent includes a portion constructed from super elastic material.

29. An apparatus as recited in claim 13 wherein said coating includes material selected from the group consisting of anti-microbial and pharmaceutical drugs.

30. An apparatus as recited in claim 13 wherein said coating includes material selected from the group consisting of therapeutic agents, anti-inflammatory active agents, genes, vectors, vaccines, biological agents, cancer treatment drugs and radioactive isotopes.

31. An apparatus as recited in claim 1 wherein said tube includes slotted end sections and a slotted middle section to form separated ribs.

32. An apparatus as recited in claim 31 wherein each said rib is creased radially outward to provide a narrow longitudinally extending surface.
